(19) United States
(12) Patent Application Publication
(10) Pub. No.: US 2017/0043047 A1
(43) Pub. Date: Feb. 16, 2017

(54) VOLATILE COMPOSITION DISPENSER

(71) Applicant: The Procter & Gamble Company, Cincinnati, OH (US)

(72) Inventors: Wolfram BECK, Singapore (SG); Lifang NIU, Singapore (SG); Herman William MEYER, West Harrison, OH (US); Keri Marsh ARINSMIER, Cincinnati, OH (US); Michael Sean FARRELL, Terrace Park, OH (US); Stefano DEFLORIAN, Trento (IT)

(21) Appl. No.: 15/233,994

(22) Filed: Aug. 11, 2016

Related U.S. Application Data
(60) Provisional application No. 62/204,449, filed on Aug. 13, 2015.

(51) Int. Cl. A61L 9/12

(52) U.S. Cl. CPC A61L 9/12

(57) ABSTRACT

The invention relates to a volatile composition dispenser. The volatile composition dispenser comprises a sealed reservoir containing a volatile composition, a membrane surrounding at least part of the sealed reservoir, and a rupture mechanism formed of a single piece of plastic located between the membrane and the sealed reservoir. The rupture mechanism comprises a support, and a movable portion attached to the support at a proximal end of the movable portion by at least one connection member. The connection member has a depth less than the depth of the movable portion and/or a width less than the width of the movable portion. One or more rupture elements are located at a distal end of the movable portion, wherein the distal end of the movable portion is movable relative to the support upon activation. Upon activation of the rupture mechanism, the one or more rupture elements are moved into contact with the reservoir such that they puncture the reservoir to release the volatile composition.

### FIELD OF THE INVENTION

The invention relates to a volatile composition dispenser and in particular with a rupture mechanism for providing a deflection within the volatile composition dispenser when the volatile composition dispenser is pressed. More particularly, the invention relates to an apparatus for delivering a volatile material comprising the volatile composition dispenser and a holder and a method of attaching the volatile composition dispenser to a holder for delivering a volatile material.

### BACKGROUND OF THE INVENTION

Systems for delivering volatile materials to the atmosphere are well known in the art. Such systems include insect repellents, air fresheners, malodor removal agents, or the like, and function by evaporating a volatile material into a space to deliver a variety of benefits such as air freshening or malodor removal. Typically, before activation of the air freshener device, a reservoir holding the volatile composition is sealed by an impermeable membrane. Upon activation, the impermeable substrate is either punctured or removed to release the volatile composition such that it comes into contact with a permeable membrane located adjacent the reservoir.

US Patent Application Publication No. 2011/0180621A1 in the name of The Procter & Gamble Company describes an apparatus for delivering a volatile material to the atmosphere in a continuous manner. The apparatus comprises a replaceable refill cartridge that is activated to release the volatile material upon insertion into a housing designed to be kept and reused.

An alternative way of activating a volatile composition dispenser is described in PCT Publication No. WO 2010/121039 A2. The dispenser comprises a container in which a volatile composition is held and that includes a rupture element, and a housing in which the container is received and that comprises a cam comprising a camming surface. The camming surface is configured to move at least a portion of a rupture element until it punctures the container to release at least a portion of the volatile composition that subsequently evaporates and exits the volatile composition dispenser.

A problem associated with the above described volatile composition dispensers is the number of mechanisms required to activate the products. First, the cost to manufacture and assemble multiple pieces increases the product cost. Second, as the dispensers are generally used in a sanitary space such as the toilet, the consumers may dispose of the dispenser housing rather than reusing it, which negates the benefit of having a reusable housing. Such housing tends to be formed of material that is not recyclable, and disposal of such dispensers is not a sustainable practice as it generates more non-renewable waste which cause harm to the environment.

Therefore, there exists a need for a sustainable design of a disposable volatile material dispenser that minimizes the use of non-renewable materials, and which can be manufactured at a low cost, and that still provides a convenient and practical user experience.

### SUMMARY OF THE INVENTION

According to an embodiment, there is provided a volatile composition dispenser comprising a sealed reservoir containing a volatile composition, a membrane surrounding at least part of the sealed reservoir and a rupture mechanism formed of a single piece of plastic located between the membrane and the sealed reservoir. The rupture mechanism comprises a support and a movable portion spaced apart from the support along a longitudinal direction and attached to the support at a proximal end of the movable portion by at least one connection member. The connection member has a depth less than the depth of the movable portion and/or a width less than the width of the movable portion. One or more rupture elements are located at a distal end of the movable portion, wherein the distal end of the movable portion is movable relative to the support upon activation. Upon activation of the rupture mechanism, the one or more rupture elements are moved into contact with the reservoir such that they puncture the reservoir to release the volatile composition.

The one or more connection members provide a hinge about which the movable portion can be moved relative to the support. The dimensions of the connection members make it possible to vary the overall stiffness of the rupture mechanism and the force required to achieve the necessary deflection for activating the rupture mechanism. Furthermore, by configuring the connection member to have a depth less than the depth of the movable portion and/or a width less than a width of the movable portion, the torque required to deflect the movable portion about the connection members is reduced, which enables the length of the movable portion to be minimized. Minimizing the length of the movable portion reduces the material required for making the rupture mechanism which results in material cost savings and less waste of plastic upon disposal. This in turns facilitates use of a compact rupture mechanism and reduces the overall cost for such a volatile composition dispenser.

The movable portion may comprise a first surface positioned adjacent the reservoir and a second surface facing away from the reservoir, wherein the second surface comprises a raised surface configured to receive force during activation of the rupture mechanism.

A rupture mechanism having such a raised surface can be used in a volatile composition dispenser of an apparatus for delivering a volatile material whereby the apparatus is hand-activated or device-activated. By “hand-activated”, it is meant to include actuation of the apparatus controlled by a user. By “device-activated”, it is meant to include actuation of the apparatus controlled by a component of the device. When device-activated, the raised surface may create an interference between the volatile composition dispenser and a housing wherein, upon insertion of the volatile composition dispenser into the housing, a part of the housing pushes against the raised surface, forcing the movable portion of the rupture mechanism to puncture the reservoir. Likewise, when hand-activated, the raised surface may provide a tactile signal to a user about where to push the rupture mechanism to activate the dispenser and may decrease the distance the user has to press the movable portion of the rupture mechanism. Therefore, the volatile composition dispenser can both enable ease of use and is suitable for accommodating apparatus of different designs.

The movable portion may be configured to, upon activation of the rupture mechanism, move from a first at rest position substantially parallel to the support, about the connection member to a second active position in which the rupture elements puncture the reservoir.

Where a raised surface is provided, it may be configured to become inverted when the movable portion arrives at the second active position, thus providing tactile feedback to a user that the volatile composition dispenser is activated.

The rupture mechanism may further comprise at least one stopping member configured to prevent movement of the movable portion beyond the second active position.

An advantage of the stopping member is that it provides a tactile feedback to a user to stop pressing and prevents the rupture elements from breaking the reservoir more than the amount required for the desired release of the volatile composition.

The stopping member may comprise at least one protrusion extending substantially in parallel to the movable portion and having a substantially flat surface facing the reservoir, wherein the protrusion prevents motion of the movable portion beyond the second active position.

An advantage of the protrusion is to minimize deflection of the movable portion and to prevent motion of the movable portion beyond the second active position.

The rupture mechanism may additionally or alternatively comprise at least one frangible member connecting the distal end of the movable portion to the support, wherein upon activation of the rupture mechanism, the frangible member breaks to allow movement of the movable portion from the at rest position. This may function as a safety mechanism to prevent accidental activation of the rupture mechanism (for example, when the volatile dispenser is in transit or by small children), and/or may provide a tactical signal to a user that the volatile dispenser has been activated and/or may help generate the required force to rupture the reservoir.

According to an embodiment, there is an apparatus for delivering a volatile material comprising:
a volatile composition dispenser comprising a sealed reservoir containing a volatile composition, a membrane surrounding at least part of the sealed reservoir and a rupture mechanism formed of a single piece of plastic located between the membrane and the sealed reservoir; and
a holder configured for receiving the volatile composition dispenser, and having at least one opening for allowing evaporation of the volatile composition upon activation.

The rupture mechanism comprises a support and a movable portion spaced apart from the support along a longitudinal direction and attached to the support at a proximal end of the movable portion by at least one connection member. The connection member has a depth less than a depth of the movable portion and/or a width less than the width of the movable portion. One or more rupture elements are located at a distal end of the movable portion, wherein the distal end of the movable portion is movable relative to the support upon activation. Upon activation of the rupture mechanism, the one or more rupture elements are moved into contact with the reservoir such that they puncture the reservoir to release the volatile composition.

The holder may be made of a foldable material. The foldable material may enable ease of storage and transportation of the holder prior to assembly.

Alternatively, the holder may be a housing comprising:
a first end wall;
a second end wall;
a first side wall;
a second side wall; and
a base; wherein the second end wall is arranged adjacent the membrane of the volatile composition dispenser, and comprises a contact surface on which an activation force is receivable.

By having a contact surface on which an activation force is receivable, the housing enables the apparatus to be hand-activated.

The first side wall may be inclined at a first angle with respect to the first end wall and the second side wall is inclined at a second angle with respect to the second end wall so as to define a substantially trapezoidal shape in the base. The trapezoidal shape of the base enables the housing to be supported on a surface where the apparatus is placed for delivering the volatile composition.

According to an embodiment, there is a method of attaching a volatile composition dispenser to a holder for delivering a volatile material, the method comprising:
positioning an edge located at a proximal end of a volatile composition dispenser into a top end of a holder; and
sealing the holder and the volatile composition dispenser at the top end of the holder;
wherein the volatile composition dispenser comprises:
a sealed reservoir containing a volatile composition;
a membrane surrounding at least part of the sealed reservoir;
a rupture mechanism formed of a single piece of plastic located between the membrane and the sealed reservoir;
wherein the rupture mechanism comprises:
   a) a support;
   b) a movable portion spaced apart from the support along a longitudinal direction and attached to the support at a proximal end of the movable portion by at least one connection member, the connection member having a depth less than the depth of the movable portion and/or a width less than the width of the movable portion;
   c) one or more rupture elements located at a distal end of the movable portion, wherein the distal end of the movable portion is movable relative to the support upon activation;
   wherein upon activation of the rupture mechanism, the one or more rupture elements are moved into contact with the reservoir such that they puncture the reservoir to release the volatile composition.

An advantage of the above method is that the apparatus can be assembled quickly without requiring particular user skill or strength.

The method may further comprise sliding the volatile composition dispenser in a direction along a y-axis of the holder.

According to an embodiment, there is a plastic rupture mechanism for a volatile composition dispenser comprising a sealed reservoir containing a volatile composition, the rupture mechanism comprising:
a) a support;
b) a movable portion spaced apart from the support along a longitudinal direction and attached to the support at a proximal end of the movable portion by at least one connection member, the connection member having a depth less than the depth of the movable portion and/or a width less than the width of the movable portion;
c) one or more rupture elements located at a distal end of the movable portion, wherein the distal end of the movable portion is movable relative to the support upon activation;
wherein upon activation of the rupture mechanism, the one or more rupture elements are moved into contact with the reservoir such that they puncture the reservoir to release the volatile composition.

By having the connection member connecting the movable portion to the support, a rupture mechanism to be customized to accommodate reservoirs of different sizes by varying the geometry of the connection member.

### BRIEF DESCRIPTION OF THE DRAWINGS

While the specification concludes with the claims particularly pointing out and distinctly claiming the invention, it is believed that the present invention will be better understood from the following description taken in conjunction with the accompanying drawings in which:

FIG. 1 shows a perspective view of a volatile composition dispenser according to an embodiment;

FIG. 2 shows an exploded assembly view of the volatile composition dispenser;

FIG. 3 shows a top view of a rupture mechanism for a volatile composition dispenser according to an embodiment;

FIG. 4 shows a side view of the rupture mechanism of FIG. 3;

FIG. 5 shows a section view of the rupture mechanism of FIG. 4 along an A-A section line;

FIG. 6 shows a top perspective view of a rupture mechanism having a plurality of stopping members arranged adjacent rupture elements according to an embodiment;

FIG. 7A and FIG. 7B show section views of the rupture mechanism of FIG. 6 in a partial assembly of a volatile composition dispenser before activation and upon activation;

FIG. 8 shows a top perspective view of a rupture mechanism having a plurality of stopping members arranged away from rupture elements according to an embodiment;

FIG. 9 shows a top perspective view of a rupture mechanism having a plurality of stopping members arranged away from rupture elements according to an embodiment;

FIG. 10A shows a top view of a rupture mechanism configured for use in different designs of apparatus for delivering a volatile material according to an embodiment;

FIG. 10B shows a section view of the rupture mechanism of FIG. 10A along a B-B section line.

FIG. 11 shows a top perspective view of a partial assembly of a volatile composition dispenser comprising a rupture mechanism having a least one frangible member according to an embodiment;

FIG. 11A shows a detailed view of FIG. 11;

FIG. 12 shows a top perspective view of a rupture mechanism comprising a frangible member arranged for activation in a first orientation according to an embodiment;

FIG. 13 shows a top perspective view of a rupture mechanism comprising a plurality of frangible members and configured for activation in a second orientation different from the first orientation;

FIG. 14A shows a top perspective view of a partial assembly of a volatile composition dispenser comprising a rupture mechanism having slidable surfaces according to an embodiment;

FIG. 14B shows a section view of the rupture mechanism of FIG. 14A;

FIG. 15A shows a side section view of a volatile composition dispenser according to an embodiment in a first at rest position;

FIGS. 15B and 15C show side section views of the volatile composition dispenser in different second active positions upon applying different forces on the volatile composition dispenser;

FIG. 16 shows an exploded assembly of an apparatus for delivering a volatile material according to an embodiment;

FIG. 17A shows a front view of a housing for an apparatus for delivering a volatile material according to an embodiment;

FIG. 17B shows a back view of the housing of FIG. 17A;

FIG. 17C shows a bottom view of the housing of FIG. 17A;

FIG. 18A shows an illustration of an apparatus for delivering a volatile material before activation;

FIG. 18B shows an illustration of the apparatus of FIG. 18A during activation;

FIG. 19 shows a top section view of a volatile composition dispenser in a first apparatus for delivering a volatile material;

FIG. 20 shows a top section view of a volatile composition dispenser in a second apparatus for delivering a volatile material; and

FIG. 21 shows a cardboard blank for making a housing for an apparatus for delivering a volatile material according to an embodiment.

### DETAILED DESCRIPTION OF THE INVENTION

Various embodiments will now be described to provide an overall understanding of the principles of the structure, function, manufacture, and use of the apparatuses and methods disclosed herein. One or more examples of these embodiments are illustrated in the accompanying drawings. Those of ordinary skill in the art will understand that the apparatuses and methods specifically described herein and illustrated in the accompanying drawings are non-limiting example embodiments and that the scope of the various embodiments of the present disclosure are defined solely by the claims. The features illustrated or described in connection with one example embodiment may be combined with the features of other example embodiments. Such modifications and variations are intended to be included within the scope of the present disclosure.

### Volatile Composition Dispenser

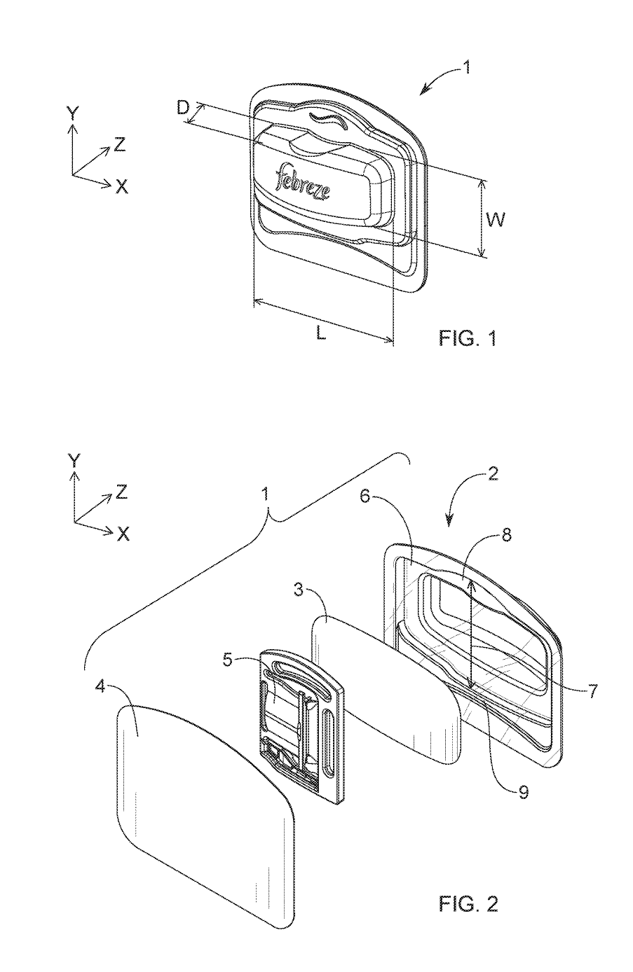

FIG. 1 shows an embodiment of a volatile composition dispenser 1 of the present invention. FIG. 2 shows an exploded view of the internal mechanism. The volatile composition dispenser 1 comprises a reservoir 2 for containing a volatile composition, a substrate 3 positioned adjacent the reservoir 2 for sealing the reservoir 2 to form a sealed reservoir containing the volatile composition, a membrane 4 and a rupture mechanism 5 located between the membrane 4 and the substrate 3. The volatile composition dispenser 1 may comprise at least one volatile composition or material capable of being evaporated under atmospheric conditions and configured for air freshening, neutralizing odor, counteracting odor, aromatherapy, repelling insects, and/or a combination thereof.

As shown in FIG. 1, the sealed reservoir comprises a length L, width W and depth D along an x-axis, y-axis and z-axis respectively. It will be appreciated that the abovementioned dimensions and dimensions described in the following description may vary from actual measured measurements in view of curved surfaces as shown in the embodiments. It will be appreciated by a person skilled in the art that the dimensions may be measured as shown in the embodiments with measuring instruments including digital or Vernier calipers or optical probes.

For example, the sealed reservoir may have a length L of about 45 mm to about 55 mm, alternatively about 50 mm measured in the x-axis, a width W of about 15 mm to about 30 mm, alternatively about 25 mm measured in the y-axis, and a depth D of about 5 to about 15 mm, alternatively about 10 mm measured in the z-axis.

### Reservoir

Referring to FIG. 2, the reservoir 2 comprises an inner periphery 6 configured for receiving the rupture mechanism 5. Specifically, the inner periphery 6 comprises a gap 7 sized to receive the rupture mechanism 5 wherein the gap 7 has a width defined by a curved edge 8 and a bottom edge 9 opposite the curved edge 8. The reservoir 2 may be made of a laminated material configured to achieve desired characteristics of permeability of the volatile material, oxygen or water vapor. For example, the reservoir 2 may be made of a laminated material comprising a plastic material suitable for thermoforming processes such as for example, PET.

### Membrane

The membrane 4 may have a thickness of about 0.01 mm to 1 mm measured in the z-axis and may be made of a vapor permeable material capable of wicking liquid yet prevents free flow of liquid out of the membrane 4. The membrane 4 is sized and arranged to seal the reservoir 2 and the rupture mechanism 5 within the dispenser 1. For example, the membrane may have a length of about 70 mm, a width of 50 mm and a depth or thickness of about 0.01 to about 1 mm in the z-axis, alternatively about 0.15 to about 0.35 mm, alternatively about 0.3 mm.

### Substrate

The substrate 3 may be made of a rupturable material including flexible films such as a polymeric film, a flexible foil or a composite material such as metal foils such as aluminum foil, polymeric film laminates or a combination thereof. The substrate 3 may have a length of 60 mm, a width of 30 mm and a depth of 0.02 mm.

### Rupture Mechanism

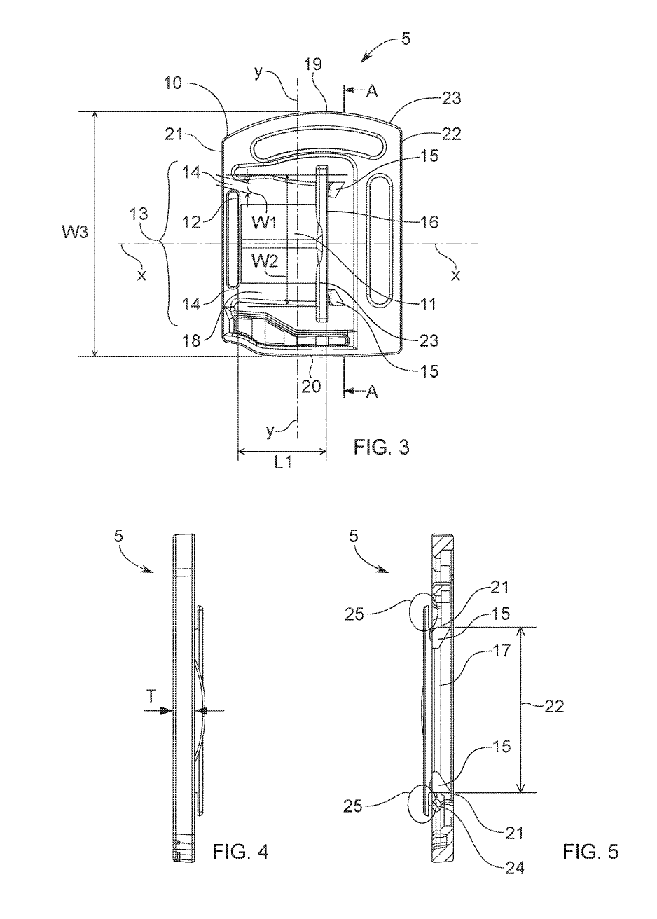

The geometry of the rupture mechanism 5 is described in detail in the following description with reference to FIGS. 3, 4 and 5. FIG. 3 shows a top view of the rupture mechanism 5 which shows a top surface facing towards the membrane 4 upon assembly to form the dispenser 1 of FIG. 1 and FIG. 2. A Cartesian coordinate system is defined with respect to the top view in which there is an x-axis and a y-axis. FIG. 4 shows a side view of the rupture mechanism 5. FIG. 5 shows a section view along an A-A section line of the rupture mechanism 5. The rupture mechanism 5 comprises a support 10 and a movable portion 11 spaced apart from the support along a longitudinal direction and attached to the support 10 at a proximal end 12 of the movable portion 11 by at least one connection member 13. The longitudinal direction is parallel to the x-axis or length of the rupture mechanism 5.

FIG. 3 shows the connection member 13 positioned with respect to or relative to a length L1 of the movable portion 11 and in the form of two connection elements 14 each having a width W1 less than a width W2 of the movable portion 11. The length L1 is measured in the x-axis. The connection elements 14 may be symmetrically positioned with respect to the width of the movable portion 11, about a center line of the movable portion 11 parallel to or along the x-axis. The width W1 and W2 are measured in the y-axis shown. The one or more connection members provide a hinge about which the movable portion can be moved relative to the support. The dimensions of the connection members make it possible to vary the overall stiffness of the rupture mechanism and the force required to achieve the necessary deflection for activating the rupture mechanism. Furthermore, by configuring the connection member to have a depth less than the depth of the movable portion and/or a width less than a width of the movable portion, the torque required to deflect the movable portion about the connection members is reduced, which enables the length of the movable portion to be minimized. Minimizing the length of the movable portion reduces the material required for making the rupture mechanism which results in material cost savings and less waste of plastic upon disposal. This in turns facilitates use of a compact rupture mechanism and reduces the overall cost for such a volatile composition dispenser.

Further, by configuring the connection elements 14 to have a depth and/or width less than a depth and/or width of the movable portion 11, a beam with non-uniform cross sections and variable stiffness is created which enable a length L1 of the movable portion 11 along the x-axis to be minimized. Specifically, as shown in FIG. 3, the connection member 13 is offset with respect to the support along the x-axis to improve the flexibility of the movable portion 11 over designs having a movable portion of similar length to length L1 or where there is no connection member 13 or the connection member is not offset. Minimizing the length L1 of the movable portion 11 reduces a plastic material used for making the rupture mechanism 5 which results in material cost savings and less waste of plastic upon disposal. It also enables the smallest size and cost for a volatile composition dispenser 1. Further, an overall stiffness of the rupture mechanism can be varied to achieve a deflection according to a desired force required for activation of the rupture mechanism 5 by pressing the rupture mechanism 5 with a finger.

One or more rupture elements 15 are located at a distal end 16 of the movable portion 11, wherein the distal end 16 of the movable portion 11 is movable relative to the support 10 upon activation. Upon activation of the rupture mechanism 5, the one or more rupture elements 15 are moved into contact with the reservoir 2 such that they puncture the substrate 3 to release the volatile material.

According to an embodiment, the movable portion 11 may be configured to move from a first at rest position substantially parallel to the support 10, about the connection elements 14 to a second active position in which the rupture elements 15 puncture the substrate 3 sealing the reservoir 2. By configuring the movable portion 11 to move about the connection elements 14, the distance between the support 10 and the movable portion 11, i.e. the offset of the connection elements 14 from the support 10 defines a lever arm which allows the user to cause a deflection or movement of the movable portion 11 based on a minimum length of the movable portion 11 and with ease of applying a force on the rupture mechanism 5.

Referring to FIG. 3 and FIG. 5, the movable portion 11 has a first surface 17 positioned adjacent the reservoir 2 and a second surface 18 facing away from the reservoir 2 upon assembly to form the volatile composition dispenser 1. Further, the rupture mechanism 5 comprises a top edge 19, a bottom edge 20, a first side edge 21 and a second side edge 22 defining an outer periphery 23 of the rupture mechanism 5. The top edge 19 may be sized and shaped to correspond to the curved edge 8 on the inner periphery 6 of the reservoir 2 (see FIG. 2). The top edge 19 and the bottom edge 20 are spaced to define a width W3 of the rupture mechanism 5. The width W3 may be determined based a distance from a tangent on the top edge 19 to a substantially flat surface on the bottom edge 20. The width W3 may have a value corresponding to a width of the gap 7 of the reservoir 2 or may be sized to fit a width of an opening of another reservoir for a volatile composition dispenser. In an embodiment, the width W3 may be about 38 to 39 mm. The top edge 19 may also function as a guiding surface for assembly of the rupture mechanism 5. Referring to FIG. 4, the movable portion 11 and the connection member 13 may have a depth or thickness T measured in the z-axis. In the above embodiment, the movable portion 11 and the connection member 13 have an uniform depth, and the width W1 of the connection member 13 is less than the width W2 of the movable portion 11. However, it will be appreciated by the person skilled in the art that the overall stiffness of the rupture mechanism can be varied or adjusted by setting the depth of the connection member 13 to be less than the depth of the movable portion 11.

FIG. 5 shows the rupture elements 15 in the form of two pin shaped elements 15 extending from the first surface 17 of the movable portion 11. Each rupture element 15 has a side surface 21 and the rupture elements 15 are spaced to define a distance 22 between the side surfaces 21. The distance 22 is measured in the y axis. The distance 22 may be configured for providing a rupture pattern of two holes in the substrate 3 upon activation to enable release of the volatile composition or material from the reservoir 2 to sufficiently wet the membrane 4. For example, the distance 22 may be about 15 to 20 mm. Specifically, the distance 22 may be about 19 to 20 mm. More specifically, the distance 22 may be about 19.2 to 19.5 mm. It will be appreciated, however, that the rupture element(s) may take many different arrangements as outlined later.

The support 10, the movable portion 11, the one or more rupture elements 15, and the connection member 13 may be a single unitary element and may be formed by a single piece of plastic. The rupture mechanism 5 may be made of plastic materials suitable for injection molding including polyolefin, polyethylene, polyester, or polypropylene (PP), preferably PP with 30% glass fiber reinforcement.

According to an embodiment, the rupture mechanism 5 may further comprise a rib 23 extending substantially in parallel to the movable portion 11 wherein the protrusion 23 has a substantially flat surface 24 facing the reservoir 2. The rib 23 increases the rigidity of the movable portion 11 thereby reducing unintended activation during storage or packing for transportation of a volatile composition dispenser. Providing the rib 23 adjacent the rupture elements 15 also reduces warpage in the movable portion 11 or bending the movable portion 11 in a plane parallel to the first or second surfaces 17, 18 when the rupture mechanism 5 is pressed upon activation.

According to an embodiment, the rupture mechanism 5 may further comprise at least one stopping member 25 configured to prevent movement of the distal end 16 of the movable portion 11 beyond the second active position. FIG. 4 shows the stopping member 25 in the form of stop elements 25 integral with the rib 23 with a stop element 25 extending along a width W2 outside a periphery of the movable portion 11.

An advantage of the stopping member 25 is that it provides a tactile feedback to a user to stop pressing and prevents the rupture elements from breaking the reservoir more than the amount required for the desired release of the volatile composition.

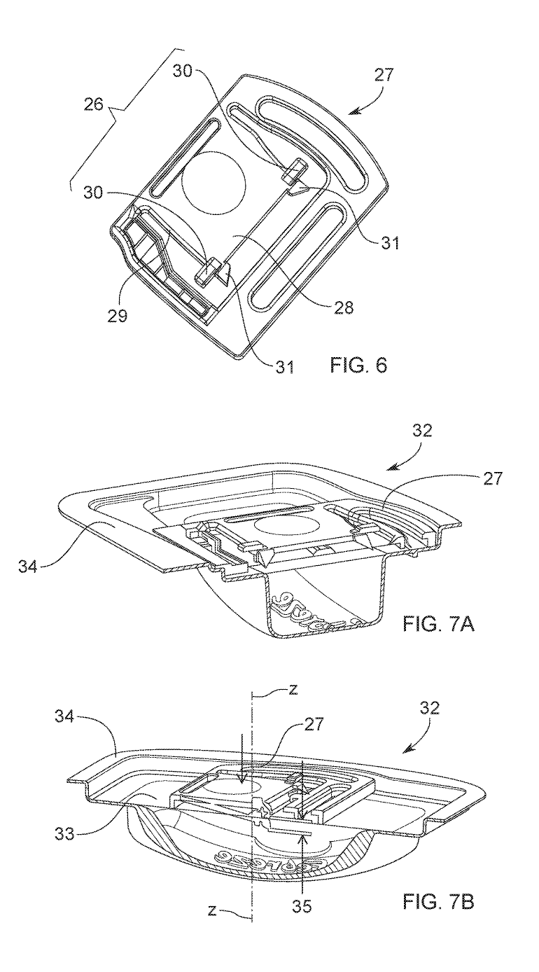

FIG. 6 shows an alternative embodiment of a stopping member 26 for a rupture mechanism 27 in which the difference from the rupture mechanism 5 is the rupture mechanism 27 does not have a rib 23. Specifically, the stopping member 26 is arranged on a surface 28 of a movable portion 29, the surface 28 facing away from the reservoir, and comprising a stop element 30 arranged adjacent at each rupture element 31.

FIG. 7A shows a section view of a partial assembly of a volatile composition dispenser 32 comprising the rupture mechanism 27 in a first at rest position. FIG. 7B shows a section view of the volatile composition dispenser 32 in a second activation position. It will be appreciated that the volatile composition dispenser 32 comprises a membrane which is not shown so as to better illustrate movement of the rupture mechanism 27 upon activation.

Referring to FIG. 7B, when the rupture mechanism 27 is depressed in a direction along the z-axis through a user pressing a membrane of the volatile composition dispenser 32 through a contact surface adjacent to the membrane. When the movable portion 29 of the rupture mechanism 27 is depressed, the movable portion 29 moves to rupture a substrate 33 sealing a reservoir 34 but does not move beyond the second active position or beyond a distance 35 upon the stopping member 26 or stop elements 30 abutting the substrate 33. The distance 35 may be a stroke distance of the rupture mechanism 27 relative to a support of the rupture mechanism 27. The distance 35 is measured in the z-axis and may be about 2.5 to 2.6 mm.

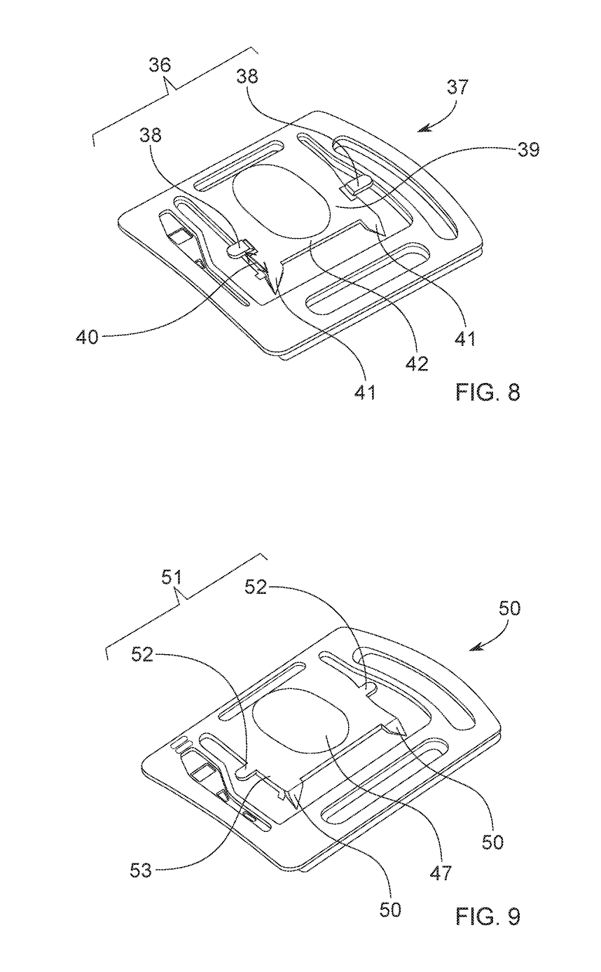

It will be appreciated, however, that a rupture mechanism may have substantially the same components but a stopping member in many different arrangements as outlined in the following description with reference to FIGS. 8 and 9. Specifically, FIG. 8 shows a stopping member 36 for a rupture mechanism 37 having a similar configuration as the rupture mechanism 27 in the form of stop elements 38 provided on a surface 39 facing away from the reservoir and arranged at a distance 40 away from rupture elements 41 for enabling a stroke distance of about 4 mm. The distance 40 is measured in the x-axis and may be about 3 mm from the rupture elements 41 along a length of a movable portion 42 of the rupture mechanism 37. The stop elements 38 may be symmetrically positioned relative to the width of the movable portion 42.

FIG. 9 shows a top perspective view of a rupture mechanism 50 having a stopping member 51 in the form of stop elements 52 extending from side surfaces 53 of a movable portion 54 according to an embodiment, and symmetrically positioned relative to the width of the movable portion 54. Specifically, the stop elements 52 are spaced from rupture elements 53 to define a distance 54 from the rupture elements 53, for example about 5 mm. This enables that the rupture mechanism 50 does not move beyond a stroke distance of 3 mm from a support 55.

Based on the above embodiments, varying a location or arrangement of a stopping member enables the stroke distance of a rupture mechanism to be varied and also prevents an unintended rupture pattern in a substrate sealing a reservoir.

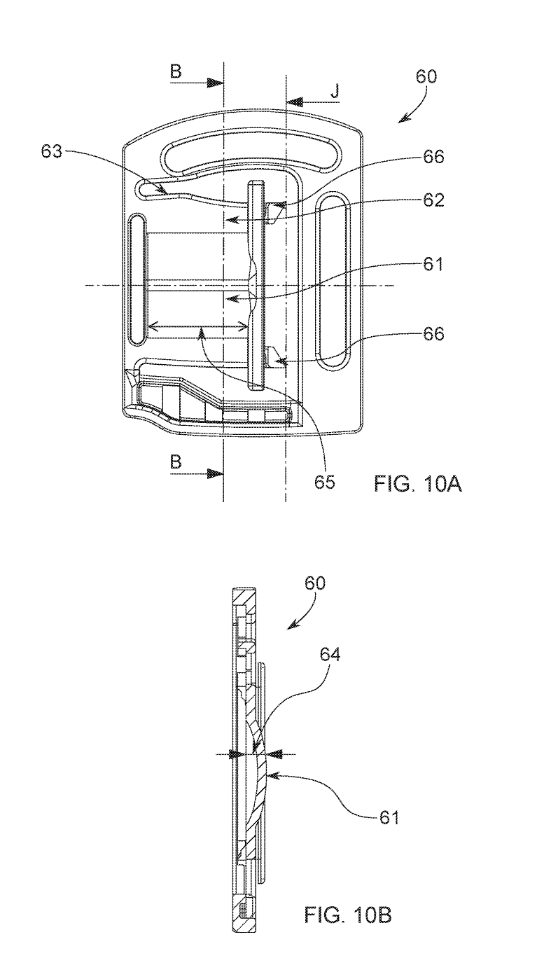

FIG. 10A shows a top view of a rupture mechanism 60 configured for use in different designs of apparatus for delivering a volatile material including known apparatus and an apparatus according to an embodiment. The rupture mechanism 60 comprises the same elements as the rupture mechanism 5. In addition, the rupture mechanism 60 has a raised surface 61 configured to receive force during activation of the rupture mechanism 60. The raised surface 61 may be arranged on a surface 62 of a movable portion 63 facing away from the reservoir upon assembly. The raised surface 61 may be configured to invert upon depression of the movable portion 63.

A rupture mechanism having such a raised surface can be used in a volatile composition dispenser of an apparatus for delivering a volatile material whereby the apparatus is hand-activated or device-activated. By “hand-activated”, it is meant to include actuation of the apparatus controlled by a user. By “device-activated”, it is meant to include actuation of the apparatus controlled by a component of the device. When device-activated, the raised surface may create an interference between the volatile composition dispenser and a housing wherein, upon insertion of the volatile composition dispenser into the housing, a part of the housing pushes against the raised surface, forcing the movable portion of the rupture mechanism to puncture the reservoir. Likewise, when hand-activated, the raised surface may provide a tactile signal to a user about where to push the rupture mechanism to activate the dispenser and may decrease the distance the user has to press the movable portion of the rupture mechanism. Therefore, the volatile composition dispenser can both enable ease of use and is suitable for accommodating apparatus of different designs.

FIG. 10B shows a section view of the rupture mechanism 60 along section line B-B. The raised surface 61 may be a protrusion 61 having a depth or height 64 relative to a support of the rupture mechanism 60. Specifically the depth or height 64 is measured in the z-axis and may be about 2 mm to 3 mm. The protrusion 61 may have a length 65 extending from a proximal end of the movable portion 63 towards the rupture elements 66 located at a distal end of the movable portion 63. The length 65 may be less than or equal to a length of the movable portion 63.

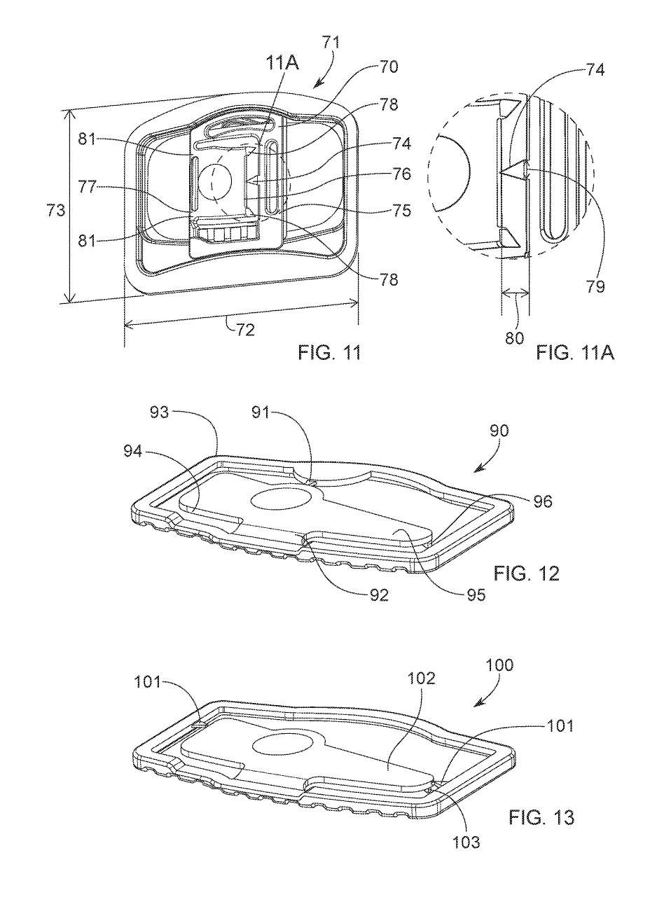

The rupture mechanism may additionally or alternatively comprise at least one frangible member connecting the distal end of the movable portion to the support, wherein upon activation of the rupture mechanism, the frangible member breaks to allow movement of the movable portion from the at rest position. This may function as a safety mechanism to prevent accidental activation of the rupture mechanism (for example, when the volatile dispenser is in transit or by small children), and/or may provide a tactical signal to a user that the volatile dispenser has been activated and/or may help generate the required force to rupture the reservoir. Different arrangements of the frangible member for a volatile composition dispenser are described below with reference to FIGS. 11 to 13.

FIG. 11 shows a top perspective view of a rupture mechanism 70 for a volatile composition dispenser 71 wherein a membrane is not shown to better illustrate the rupture mechanism 70. FIG. 11A is a detailed view of FIG. 11. The volatile composition dispenser 71 is in a horizontal orientation in which the volatile composition dispenser 71 has a length 72 along an x-axis and a width 73 along a y-axis. The rupture mechanism 70 has a frangible connection 74 extending from a support 75 and attaching the support 75 to a distal end 76 of a movable portion 77. Rupture elements 78 are provided at the distal end 76. Further, the frangible connection 74 comprises a variable width 79 along a length 80 of the frangible connection 74 and may form a substantially V shaped member with a point connected to the movable portion 77. Upon activation by applying a force on the movable portion 77, the movable portion 77 detaches from the frangible connection 74 at the distal end 76 and the movable portion 77 is movable about connection elements 81. The frangible connection 74 prevents unintended activation by allowing the movable portion 77 to be maintained in a first position in which rupture elements 78 do not puncture a substrate sealing the reservoir.

FIG. 12 shows a top perspective view of a rupture mechanism 90 configured for activation of a volatile composition dispenser configured for use in a vertical orientation opposite to the horizontal orientation described in FIG. 11. A frangible connection 91 may be disposed within the rupture mechanism 90. The rupture mechanism 90 comprises a connection member 92 for attaching a support 93 to a proximal end 94 of a movable portion 95. The connection member 92 has a width less than the width of the movable portion 95. The frangible connection 91 may be located at a position where a force is receivable on the movable portion 95 and arranged away from rupture elements 96.

FIG. 13 shows a top perspective view of another embodiment of a rupture mechanism 100 configured for activation of a volatile composition dispenser configured for use in a vertical orientation opposite to the horizontal orientation described in FIG. 11. The rupture mechanism 100 has substantially the same features as the rupture mechanism 90 and differs in having more than one frangible connection 101 and a different arrangement of the frangible connections 101. Specifically, the rupture mechanism 100 comprises a plurality of frangible connections 101 wherein each frangible connection 101 is located at a position of at least one rupture element(s) 103.

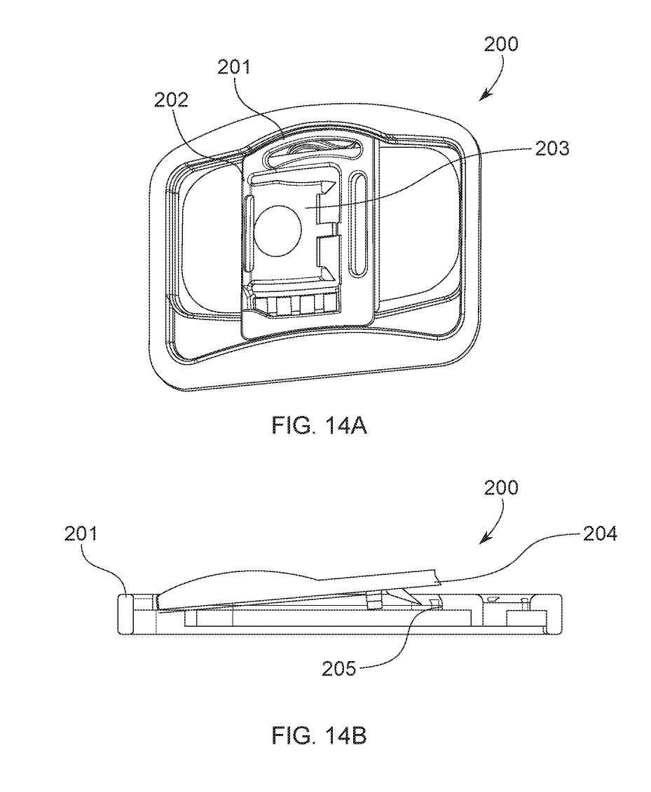

FIG. 14A shows a top view of a partial assembly of a volatile composition dispenser 200 in which a membrane is removed. FIG. 14B is a section view of the volatile composition dispenser 200 having a rupture mechanism 201 in a raised position. The rupture mechanism 201 comprises a support 202 and a movable portion 203 having a sliding surface 204 configured for sliding past a sliding surface 205 of the support 202. Upon activation by pressing the movable portion 203, the sliding surface 204 slides along the sliding surface 205 and the sliding action generates a bearing force on the surfaces 204, 205 which provides a tactile feedback to the user.

### Activation of a Volatile Composition Dispenser

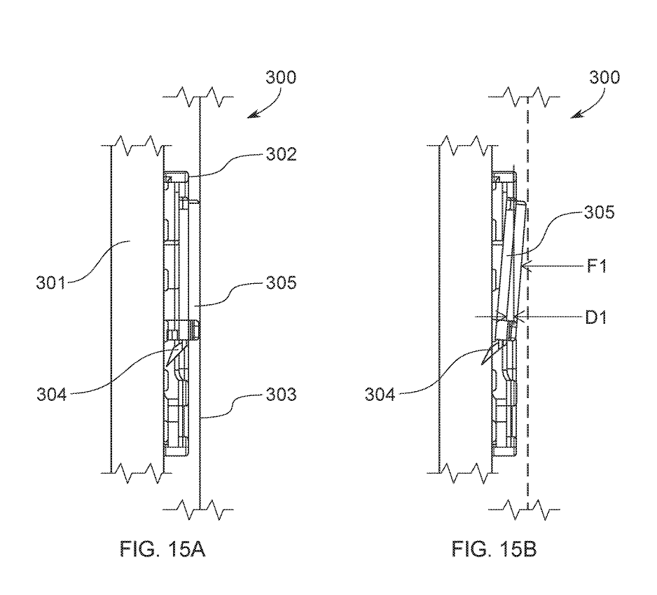

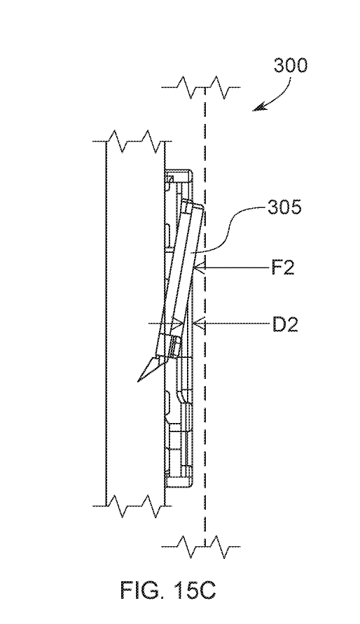

Activation of a volatile composition dispenser 300 according to an embodiment will be described in detail in the following description with respect to FIGS. 15A to 15C. The volatile composition dispenser 300 is similar in configuration to the volatile composition dispenser 1 of FIG. 1 and FIG. 2 and comprises a sealed reservoir 301, a rupture mechanism 302 and a membrane 303. FIG. 15A shows a section view of the volatile composition dispenser 300 in a first at rest position before activation. The rupture mechanism 302 is supported on the reservoir 301 through a surface of the reservoir 301 in the first at rest position wherein the rupture mechanism 302 is stationary and rupture elements 304 do not puncture the reservoir 301.

FIGS. 15B and 15C show side section views of a volatile composition dispenser 300 in different positions upon applying different forces on the volatile composition dispenser.

FIG. 15B illustrates movement of the rupture mechanism 302 through a stroke distance D1 upon activation of an activation force F1 in a direction perpendicular to a surface of the membrane 303 or a direction parallel to a z-axis. The stroke distance D1 is measured in the z-axis. Upon activation, a movable portion 305 of the rupture mechanism 302 is movable relative to a support to travel through the stroke distance D1 from the first at rest position in FIG. 15A to a second active position in which the one or more rupture elements 304 rupture the substrate 21. FIG. 15C illustrates movement of the rupture mechanism 302 through a stroke distance D2 upon activation of an activation force F2 in a direction perpendicular to a surface of the membrane 303 or a direction parallel to the z-axis. It will be appreciated that the stroke distance D1/D2 may vary according to the activation force F1/F2. For example, the activation force F to rupture the substrate 21 may include one of: 15N to 19N at the stroke distance D of 2 mm, 24N to 31N at the stroke distance D of 2.5 mm, 36N to 44N at the stroke distance D of 3 mm. The rupture mechanism 302 may be configured to bias the movable portion 305 towards the first position after activation.

### Apparatus for Delivering a Volatile Composition

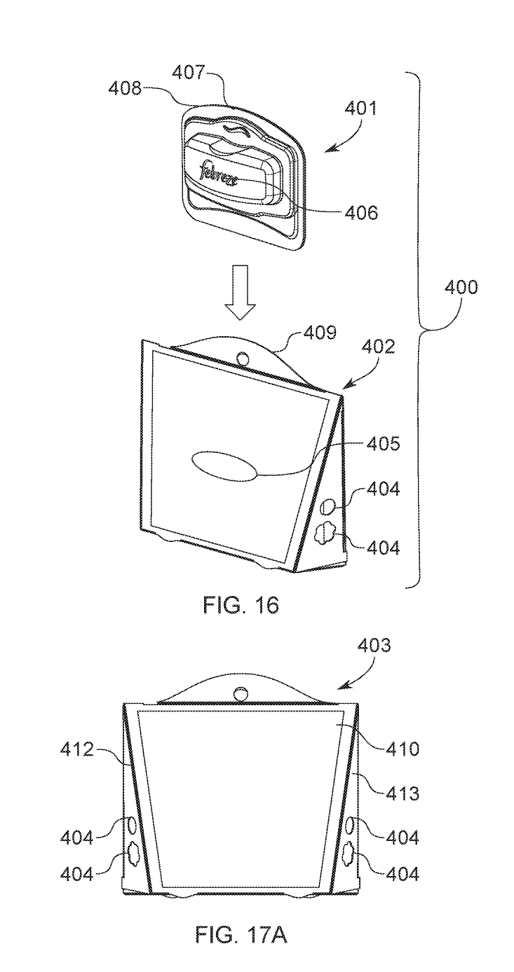

FIG. 16 shows an apparatus 400 for delivering a volatile material according to an embodiment. The apparatus 400 comprises a volatile composition dispenser 401 and a holder 402 configured for receiving the volatile composition dispenser 401. The volatile composition dispenser 401 is shown in a horizontal orientation as described in FIG. 11.

### Holder

The holder 402 may be a housing 403 sized and shaped for receiving the volatile composition dispenser 401. The holder 402 may have one or more openings 404 for allowing evaporation of the volatile composition upon activation of the apparatus 400. The holder 402 may include a window 405 configured for displaying a logo 406 printed on the sealed volatile composition dispenser 401 as a visual indicator to consumers. The volatile composition dispenser 401 may include an edge 407 located at a proximal end 408 for positioning the volatile composition dispenser 401 into a top end 409 of the housing 403. Upon assembly, the housing 403 and the volatile composition dispenser 401 may be sealed at the top end 409 to form the apparatus 400.

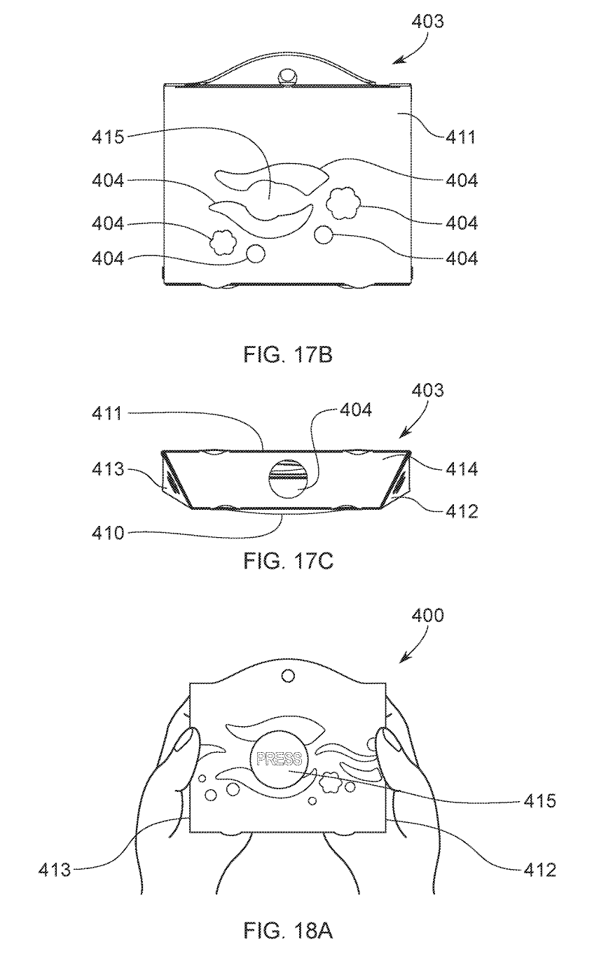

FIGS. 17A, 17B and 17C show a front view, a back view and a bottom view of the housing 403 respectively. The housing 403 comprises a first end wall 410, a second end wall 411, a first side wall 412, a second side wall 413, and a base 414. The second end wall 411 may comprise a contact surface 415 on which an activation force is receivable. In an embodiment, the housing 403 may be made of a material suitable for printing a graphic such as a logo on the housing. Visual cues may be provided on the contact surface 415 on the second end wall 411 for finger placement during use of the apparatus 400.

Referring to FIG. 17C, the first side wall 412 and the second side wall 413 may be inclined with respect to the first end wall 410 and the second end wall 411 to define a substantially trapezoidal shape in the base 414. For example, the first and second side walls 412, 413 may be respectively inclined at angles relative to the second end wall 413 so as to define a holding profile for guiding the user towards a handheld position similar to how users interact with handheld mobile devices wherein the handheld position is stable for the user to manually exert an activation force for activation of the apparatus 400 to achieve a desired result, i.e. activating the apparatus 400 in a consistent manner for delivering a volatile material.

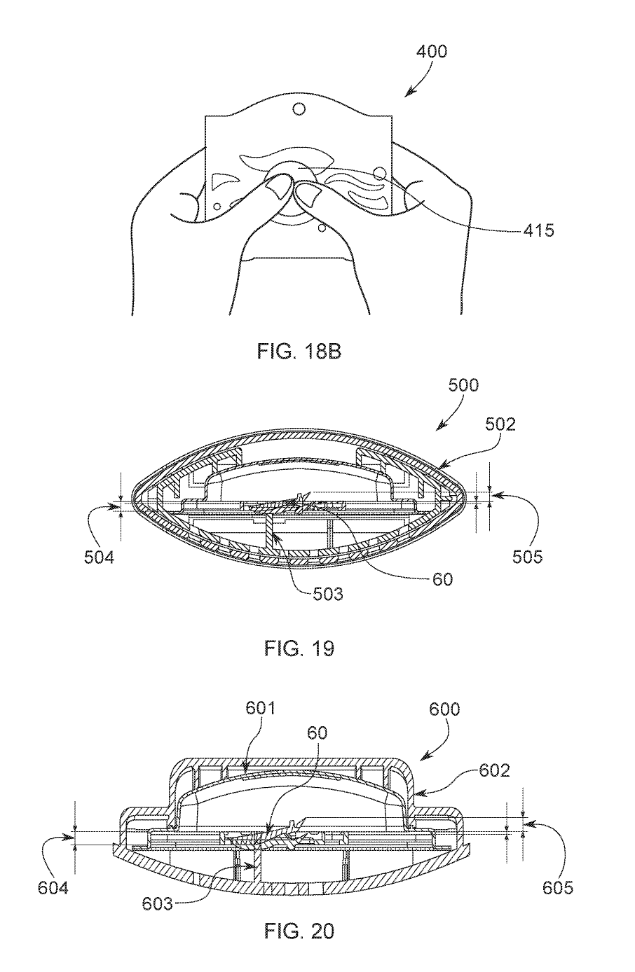

An example of the apparatus 400 in a handheld position is illustrated in FIG. 18A which shows a back view of the apparatus 400 prior to activation and FIG. 18B illustrates the apparatus 300 during activation wherein an activation force being applied on the contact surface 415 when the user presses the contact surface 415. Further, in an embodiment, the apparatus 400 may be of a size and weight configured to allow ease of activation of the apparatus 300 in a handheld position in close proximity to the body of the user.

FIG. 19 shows a top section view of a first apparatus 500 for delivering a volatile material having a volatile composition dispenser 501 comprising a rupture mechanism such as for example, a rupture mechanism 60 of FIG. 10A and FIG. 10B. The first apparatus 500 comprises a housing 502 having a notch 503 configured for an interference fit 504 with the protrusion or rib. Upon insertion of the volatile composition dispenser 401, the interference of the protrusion with the notch 503 causes the movable portion comprising rupture elements to puncture the substrate thereby activating the volatile composition dispenser 501.

FIG. 20 shows a top section view of a second apparatus 600 for delivering a volatile material having a volatile composition dispenser 601 comprising the rupture mechanism 60. The second apparatus 600 comprises a housing 602 different in design from the first housing 502 and having a notch 603 configured for an interference fit 604 with the protrusion. Upon insertion of the volatile composition dispenser 601, the interference of the protrusion with the notch 503 causes the movable portion comprising rupture elements to puncture the substrate thereby activating the second apparatus 600.

An advantage is that the volatile composition dispenser may be used as a common-use component in apparatus for delivering a volatile material to the environment whereby the apparatus have housings of different designs but the stroke distance for activating the apparatus are within the same predetermine range. Common-use parts in products increase production volumes and in-turn can provide product value through economies of scale.

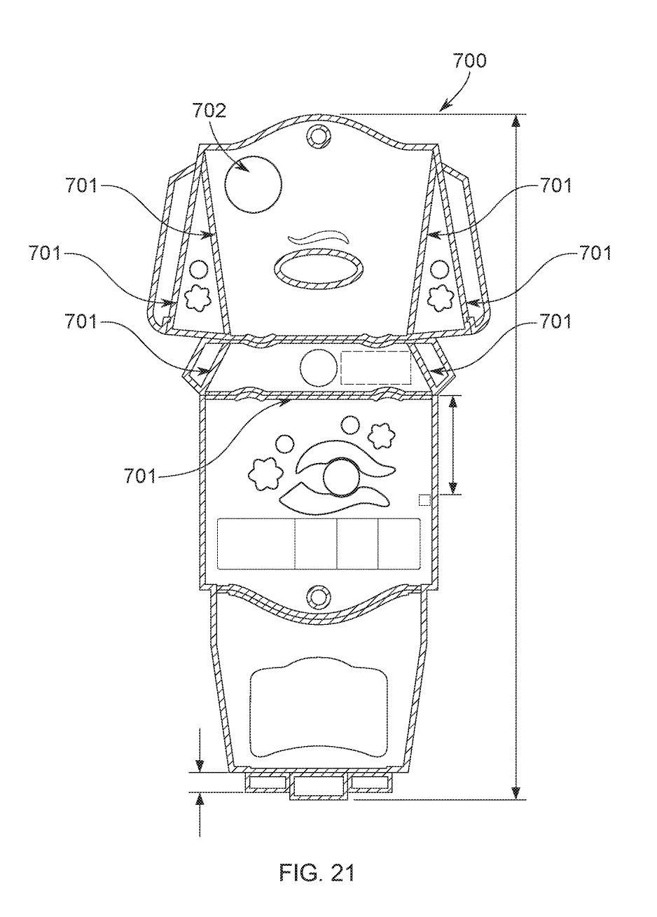

FIG. 21 illustrates an embodiment of a cardboard blank 700 suitable for making a housing (as shown in FIGS. 17A to 17C) for an apparatus for delivering a volatile composition. Folding lines 601 may be pre-formed on the cardboard blank 700, e.g. by printing, perforating or scoring. Graphics 702 may be printed on the blank 700 to provide visual cues to the consumer. The cardboard blank 700 may have an outer length of 311.71 mm such that upon assembly, the housing is sized and shaped to be held by a user for ease of activation in a handheld position.

According to an embodiment, there is a method of attaching a volatile composition dispenser to a holder for delivering a volatile material. The volatile composition dispenser may be according to any of the abovementioned embodiments. The method comprises the following steps:
a) positioning an edge located at a proximal end of a volatile composition dispenser into a top end of a holder; and
b) sealing the holder and the volatile composition dispenser at the top end of the holder.

The method may further comprise the step of sliding the sealed volatile composition dispenser in a direction along the y axis of the holder. For example, to assemble the sealed volatile composition dispenser to the housing, the sealed volatile composition dispenser may be slided in a direction along the y axis of the housing such that a microporous membrane of the sealed volatile composition dispenser is adjacent the contact surface of second end wall.

An activation force test method for demonstrating the abovementioned functionality of a volatile composition dispenser according to any one of the abovementioned embodiments is described below.

### TEST METHOD AND EXAMPLES

As a housing for an apparatus for delivering a volatile material according to an embodiment may be made of a foldable material such as paper or cardboard, therefore it will be appreciated by a person skilled in the art that an activation force of the apparatus may be determined by measuring the activation force of the volatile composition dispenser only. Accordingly, 60 samples of a volatile composition dispenser based on an embodiment of a rupture mechanism 60 as shown in FIGS. 10A and 10B were prepared and tested for an activation force of the volatile composition dispensers through different stroke distances determined for rupturing the substrate and effective wetting of the membrane upon activation. Effective wetting of the membrane may be defined by wetting of more than or equal to 50% of the membrane area in five (5) minutes upon activation.

### A) Testing System

The activation force may be measured using an electro-mechanical testing system such as for example, QTest Elite 10 system commercially available from MTS, along with a UL 283 finger probe made of polyamide and modified to not include any articulating joints such that it is in a fixed position perpendicular to a contact surface on the volatile composition dispenser. For example, the modified UL 283 finger probe comprises a round tip. It will be appreciated that the round tip is to simulate an average size of the human fingertip. Details of the UL 283 finger probe is described in Standard for Air Fresheners and Deodorizers, UL Standard 283, FIG. 10.1 (UL Mar. 31, 2004). As described in UL 283, FIG. 10.1, the radius of the finger tip is 3.5 mm; height of the finger tip is 5 mm; depth of the finger tip is 3.5 mm; depth of the finger tip is 5.8 mm. However, unlike the finger probe of UL 283, the modified UL 283 finger probe does not have any articulating joints. Instead it is in a fixed position that is perpendicular to the contact surface or the rupture mechanism in the z-axis. The contact surface also may be a top surface on the membrane adjacent to the movable portion of the rupture mechanism in each sample.

### B) Test Set-Up

Testing is conducted at a temperature of 20+/−5 degrees Celsius. The crosshead speed of the electro-mechanical testing system is set at 30 mm/min and the displacement of the modified finger probe corresponds to each specified stroke distance of the samples wherein the stroke distances are measured in the z-axis. Specifically, the volatile composition dispenser is supported on a fixture wherein the membrane is facing the modified UL finger probe, without contacting the rupture mechanism. The testing system is programmed to move the modified UL finger probe towards the rupture mechanism in the z-axis to contact a region where the stroke distance or displacement is desired for rupturing a substrate sealing the sealed reservoir. The region is within the movable portion of the rupture mechanism.

### C) Test Conditions

Details of the test conditions and success criteria for the samples are set out in the following Table 1.

TABLE 1

| Sample Size | Test Conditions | Success Criteria |
|---|---|---|
| 20 samples | Stroke distance of 2 mm | a) Activation Force |
| 20 samples | Stroke distance of 2.5 mm | Measured <50 N |
| 20 samples | Stoke distance of 3 mm | b) Greater than 50% of a |
| | | wetted area in the membrane |
| | | within 5 minutes |

### D) Test Results

Test results of the above samples are summarized in the following Tables 2, 3 and 4 and the results show that a volatile composition dispenser having a rupture mechanism according to the present invention may be activated with an activation force of less than 50N through the stroke distances ranging from 1.6 mm to 3 mm to achieve effective wetting of the membrane.

TABLE 2
Activation Force Test - Test with Round Tip

| | | | | Membrane | |
|---|---|---|---|---|---|
| | | Force | | Wetted Area | |
| Sample | Stroke | Peak | Displacement | % in 5 minutes | Overall |
| No. | Distance | (N) | (mm) | >50% ? | Result |
| #1 | | 17.0 | 2.0 | Yes | OK |
| #2 | | 16.8 | 1.6 | Yes | OK |
| #3 | | 15.9 | 1.8 | Yes | OK |
| #4 | | 17.1 | 1.9 | Yes | OK |
| #5 | | 17.0 | 2.0 | Yes | OK |
| #6 | | 17.4 | 1.8 | Yes | OK |
| #7 | | 17.6 | 2.0 | Yes | OK |
| #8 | | 17.8 | 1.9 | Yes | OK |
| #9 | | 18.6 | 1.7 | Yes | OK |
| #10 | | 16.9 | 2.0 | Yes | OK |
| #11 | 2.0 mm | 16.3 | 1.8 | Yes | OK |
| #12 | | 19.0 | 2.0 | Yes | OK |
| #13 | | 18.4 | 2.0 | Yes | OK |
| #14 | | 17.1 | 2.0 | Yes | OK |
| #15 | | 15.4 | 1.9 | Yes | OK |
| #16 | | 17.7 | 2.0 | Yes | OK |
| #17 | | 16.7 | 2.0 | Yes | OK |
| #18 | | 16.5 | 1.7 | Yes | OK |
| #19 | | 16.0 | 1.9 | Yes | OK |
| #20 | | 16.9 | 1.9 | Yes | OK |
| Average | — | 17.1 | 1.9 | — | — |
| Stdev | — | 0.9 | 0.1 | — | — |
| Max | — | 19.0 | 2.0 | — | — |
| Min | — | 15.4 | 1.6 | — | — |

As shown in the above Table 2, the activation force to rupture the substrate ranges from 15.4N to 19.0N at the stroke distance of 2 mm.

TABLE 3
Activation Force Test - Test with Round Tip

| | | | | Membrane | |
|---|---|---|---|---|---|
| | | | | Wetted Area | |
| | | | | % in | |
| Sample | Stroke | Force | Displacement | 5 minutes | Overall |
| No. | Distance | Peak (N) | (mm) | >50%? | Result |
| #21 | 2.5 mm | 26.7 | 2.5 | Yes | OK |
| #22 | | 27.2 | 2.5 | Yes | OK |
| #23 | | 27.3 | 2.5 | Yes | OK |
| #24 | | 27.8 | 2.5 | Yes | OK |
| #25 | | 26.6 | 2.5 | Yes | OK |
| #26 | | 27.1 | 2.5 | Yes | OK |
| #27 | | 27.0 | 2.5 | Yes | OK |
| #28 | | 28.3 | 2.5 | Yes | OK |
| #29 | | 27.1 | 2.5 | Yes | OK |
| #30 | | 25.8 | 2.5 | Yes | OK |
| #31 | | 30.6 | 2.5 | Yes | OK |
| #32 | | 27.5 | 2.5 | Yes | OK |
| #33 | | 26.1 | 2.5 | Yes | OK |
| #34 | | 24.6 | 2.5 | Yes | OK |
| #35 | | 28.1 | 2.5 | Yes | OK |
| #36 | | 27.3 | 2.5 | Yes | OK |
| #37 | | 30.9 | 2.5 | Yes | OK |
| #38 | | 27.3 | 2.5 | Yes | OK |
| #39 | | 27.8 | 2.5 | Yes | OK |
| #40 | | 30.1 | 2.5 | Yes | OK |
| Average | — | 27.6 | 2.5 | — | — |
| Stdev | — | 1.5 | 0.0 | — | — |
| Max | — | 30.9 | 2.5 | — | — |
| Min | — | 24.6 | 2.5 | — | — |

As shown in the above Table 3, an activation force to rupture the substrate is 24.6N to 30.9N at the stroke distance of 2.5 mm.

TABLE 4
Activation Force Test - Test with Round Tip

| | | | | Membrane | |
|---|---|---|---|---|---|
| | | | | Wetted Area | |
| | | | | % in | |
| Sample | Stroke | Force | Displacement | 5 minutes | Overall |
| No. | Distance | Peak (N) | (mm) | >50% ? | Result |
| #41 | 3.0 mm | 38.1 | 3.0 | Yes | OK |
| #42 | | 39.8 | 3.0 | Yes | OK |
| #43 | | 38.0 | 3.0 | Yes | OK |
| #44 | | 38.0 | 3.0 | Yes | OK |
| #45 | | 36.4 | 3.0 | Yes | OK |
| #46 | | 40.9 | 3.0 | Yes | OK |
| #47 | | 38.4 | 3.0 | Yes | OK |
| #48 | | 38.7 | 3.0 | Yes | OK |
| #49 | | 37.6 | 3.0 | Yes | OK |
| #50 | | 40.1 | 3.0 | Yes | OK |
| #51 | | 39.5 | 3.0 | Yes | OK |
| #52 | | 38.1 | 3.0 | Yes | OK |
| #53 | | 36.5 | 3.0 | Yes | OK |
| #54 | | 39.4 | 3.0 | Yes | OK |
| #55 | | 41.9 | 3.0 | Yes | OK |
| #56 | | 40.8 | 3.0 | Yes | OK |
| #57 | | 43.3 | 3.0 | Yes | OK |
| #58 | | 39.6 | 3.0 | Yes | OK |
| #59 | | 41.2 | 3.0 | Yes | OK |
| #60 | | 41.4 | 3.0 | Yes | OK |
| Average | — | 39.4 | 3.0 | — | — |
| Stdev | — | 1.8 | 0.0 | — | — |
| Max | — | 43.3 | 3.0 | — | — |
| Min | — | 36.4 | 3.0 | — | — |

As shown in the above Table 4, an activation force to rupture the substrate is 36.4N to 43.3N at the stroke distance of 3 mm.

It can be seen from the embodiments described in the above that an advantage of a rupture mechanism having a connection member according to the present invention is that the connection member enables the movable portion to be mechanically isolated from the support such that the movable portion may be suspended within an inner periphery of the rupture mechanism, and the movable portion is movable from a first at rest position substantially parallel to the support, about the connection member to a second active position in which the rupture elements puncture the reservoir. Further, the connection member enables minimizing a material for making the rupture mechanism thereby resulting in savings in material costs and less waste of non-renewal material upon disposal.

The rupture mechanism eliminates the need for external plastic actuators or components and reduces the number of components used for an apparatus for delivering a volatile material resulting in cost savings and ease of manufacturing as a reduced number of components needs to be stocked and tracked in production. In summary, the rupture mechanism enables a disposable apparatus for delivering a volatile material which is more sustainable from both environmental and manufacturing cost perspectives.

Further, as the movable portion is not fixed directly to the support, the support is not restricted by a design of the movable portion and may be configured in many different shapes and sizes adaptable for volatile composition assemblies having reservoirs sized for containing different volatile material volumes. Further, the support also may be designed to form an integral frame or comprise individual support elements. The integral frame may be of an elongate shape, an elliptical shape or shaped to fit an interior of a housing.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A volatile composition dispenser comprising:
a sealed reservoir containing a volatile composition;
a membrane surrounding at least part of the sealed reservoir;
a rupture mechanism formed of a single piece of plastic located between the membrane and the sealed reservoir;
wherein the rupture mechanism comprises:
a) a support;
b) a movable portion spaced apart from the support along a longitudinal direction and attached to the support at a proximal end of the movable portion by at least one connection member, the connection member having a depth less than the depth of the movable portion and/or a width less than the width of the movable portion; and
c) one or more rupture elements located at a distal end of the movable portion, wherein the distal end of the movable portion is movable relative to the support upon activation;
wherein upon activation of the rupture mechanism, the one or more rupture elements are moved into contact with the reservoir such that they puncture the reservoir to release the volatile composition.

2. A volatile composition dispenser as claimed in claim 1, wherein, upon activation of the rupture mechanism, the movable portion is configured to move from a first at rest position substantially parallel to the support about the connection member to a second active position in which the rupture elements puncture the reservoir

3. A volatile composition dispenser as claimed in claim 1, wherein the movable portion has a first surface positioned adjacent the reservoir and a second surface facing away from the reservoir, wherein the second surface comprises a raised surface configured to receive force during activation of the rupture mechanism.

4. A volatile composition dispenser as claimed in claim 3, wherein the raised surface is arranged to become inverted when the movable portion arrives at the second active position.

5. A volatile composition dispenser as claimed in claim 2, wherein the rupture mechanism further comprises at least one stopping member to prevent movement of the movable portion beyond the second active position.

6. A volatile composition dispenser as claimed in claim 2, wherein the rupture mechanism further comprises at least one protrusion extending substantially in parallel to the movable portion and having a substantially flat surface facing the reservoir, wherein the protrusion prevents motion of the movable member beyond the second active position.

7. A volatile composition dispenser as claimed in claim 2, wherein the rupture mechanism further comprises at least one frangible member connecting the distal end of the movable portion to the support, wherein upon activation of the rupture mechanism, the frangible member breaks to allow movement of the movable portion from the at rest position.

8. A volatile composition dispenser according to claim 2, wherein a deflection of the movable portion relative to the support from the first at rest position to the second active position is one of: 2 mm wherein the activation force is 15N to 19N, 2.5 mm wherein the activation force is 24N to 31N, 3 mm wherein the activation force is 36N to 44N; wherein the activation force is applied in a direction along a z-axis of the volatile composition dispenser at a speed of 30 mm/minute.

9. The volatile composition dispenser according to claim 1, wherein the volatile composition dispenser is sized and shaped to be receivable in a holder configured for dispensing a volatile composition upon activation in a handheld position.

10. An apparatus for delivering a volatile material comprising:
a volatile composition dispenser according to claim 1; and
a holder configured for receiving the volatile composition dispenser, and having at least one opening for allowing evaporation of the volatile composition upon activation.

11. The apparatus according to claim 10, wherein the holder is a housing comprising:
a first end wall;
a second end wall;
a first side wall;
a second side wall; and
a base; wherein the second end wall is arranged adjacent the membrane of the volatile composition dispenser, and comprises a contact surface on which an activation force is receivable.

12. The apparatus according to claim 10, wherein the holder is made of a foldable material.

13. The apparatus according to claim 11, wherein the first side wall is inclined at a first angle with respect to the first end wall and the second side wall is inclined at a second angle with respect to the second end wall so as to define a substantially trapezoidal shape in the base.

14. A method of attaching a volatile composition dispenser to a holder for delivering a volatile material, the method comprising:
positioning an edge located at a proximal end of a volatile composition dispenser into a top end of a holder; and
sealing the holder and the volatile composition dispenser at the top end of the holder;
wherein the volatile composition dispenser comprises:
a sealed reservoir containing a volatile composition;
a membrane surrounding at least part of the sealed reservoir;
a rupture mechanism formed of a single piece of plastic located between the membrane and the sealed reservoir;
wherein the rupture mechanism comprises:
a) a support;
b) a movable portion spaced apart from the support along a longitudinal direction and attached to the support at a proximal end of the movable portion by at least one connection member, the connection member having a depth less than the depth of the movable portion and/or a width less than the width of the movable portion; and
c) one or more rupture elements located at a distal end of the movable portion, wherein the distal end of the movable portion is movable relative to the support upon activation;
wherein upon activation of the rupture mechanism, the one or more rupture elements are moved into contact with the reservoir such that they puncture the reservoir to release the volatile composition.

15. The method of claim 14, further comprising sliding the volatile composition dispenser in a direction along a y-axis of the holder.

16. A plastic rupture mechanism for a volatile composition dispenser comprising a sealed reservoir containing a volatile composition, the rupture mechanism comprising:
a) a support;
b) a movable portion spaced apart from the support along a longitudinal axis and attached to the support at a proximal end of the movable portion by at least one connection member, the connection member having a depth less than the depth of the movable portion and/or a width less than the width of the movable portion; and
c) one or more rupture elements located at a distal end of the movable portion,
wherein the distal end of the movable portion is movable relative to the support upon activation;
wherein upon activation of the rupture mechanism, the one or more rupture elements are moved into contact with the reservoir such that they puncture the reservoir to release the volatile composition.

17. A plastic rupture mechanism as claimed in claim 16, wherein, upon activation of the rupture mechanism, the movable portion is configured to move from a first at rest position substantially parallel to the support, about the connection member to a second active position in which the rupture elements puncture the reservoir.

18. A plastic rupture mechanism as claimed in claim 16, wherein the movable portion has a first surface positioned adjacent the reservoir and a second surface facing away from the reservoir, wherein the second surface comprises a raised surface configured to receive force during activation of the rupture mechanism.

19. A plastic rupture mechanism as claimed in claim 18, wherein the raised surface is arranged to become inverted when the movable portion arrives at the second active position.

20. A plastic rupture mechanism as claimed in claim 17, wherein the rupture mechanism further comprises at least one stopping member to prevent movement of the movable portion beyond the second active position.
